(19) United States
(12) Patent Application Publication
(10) Pub. No.: US 2024/0398845 A1
(43) Pub. Date: Dec. 5, 2024

(54) USE OF SGLT-2 INHIBITORS FOR THE PREVENTION AND/OR TREATMENT OF CARDIAC DISEASES IN NON-HUMAN MAMMALS EXCLUDING FELINES, IN PARTICULAR CANINES

(71) Applicant: BOEHRINGER INGELHEIM VETMEDICA GMBH, Ingelheim am Rhein (DE)

(72) Inventors: Carla KROH, Oestrich-Winkel (DE); Ingo Ulrich LANG, Ingelheim am Rhein (DE); José MATALLO, Frankfurt am Main (DE)

(21) Appl. No.: 18/578,118

(22) PCT Filed: Jul. 26, 2022
(86) PCT No.: PCT/EP2022/070898

§ 371 Date: Jan. 10, 2024

(30) Foreign Application Priority Data
Jul. 28, 2021 (EP) 21188311.1

(51) Int. Cl. A61K 31/7056; A61K 31/7034; A61K 31/7048; A61P 9/04

(52) U.S. Cl. CPC A61K 31/7056; A61K 31/7034; A61K 31/7048; A61P 9/04

(57) ABSTRACT

The present invention is directed to the use of one or more SGLT-2 inhibitors or pharmaceutically acceptable forms thereof for the prophylaxis and/or treatment of one or more cardiac diseases in a non-human mammal/non-human mammal patient excluding a feline, in particular a canine/canine patient.

### FIELD OF THE INVENTION

The invention relates to the field of medicine, in particular to the field of veterinary medicine. The invention relates to the use of SGLT-2 inhibitors or a pharmaceutically acceptable form thereof in the prevention and/or treatment of cardiac diseases in a non-human mammal excluding a feline, in particular a canine.

### BACKGROUND INFORMATION

Canine (myxomatous) mitral-valve disease [(M)MVD] and dilated cardiomyopathy (DCM) are the most common cardiovascular conditions in dogs and the most frequent cause of canine heart failure. Additionally, aortic stenosis, typically caused by a ridge or ring of fibrotic tissue in the subaortic region (subaortic stenosis), is a common congenital defect of large breed dogs. Those conditions have specific pathophysiology, but are characterized by a reduced pump capacity, increased muscular effort and energy imbalance, finally resulting in heart failure. In people, beneficial cardiovascular effects under the use of SGLT2-Inhibitors have been observed. However, the direct effect on the heart remains unknown. Also, the pathology of cardiac disease in humans (coronary disease, stroke, infarction) differs significantly from the pathologies observed in dogs [(M)MVD, DCM]. The American College of Veterinary Internal Medicine (ACVIM) consensus statement provides specific classification and treatment references for (M)MVD. Although the guideline targets (M)MVD the classifications are commonly used for other cardiac diseases, like DCM. The reference defines the different stages of canine cardiac disease as below:
Stage A: dogs are at higher-than-average risk for developing heart failure, but without any apparent structural abnormality (i.e., no audible heart murmur) at the time of examination.
Stage B: dogs in Stage B have a structural abnormality [e.g., the presence of (M)MVD], but have never had clinical signs of heart failure associated with their disease. Stage B is divided into: Stage B1: describes asymptomatic dogs that have no radiographic or echocardiographic evidence of cardiac remodelling in response to their (M)MVD, as well as those in which remodelling changes are present, but not severe enough to meet current clinical trial criteria that have been used to determine that initiating treatment is warranted.
Stage B2: refers to asymptomatic dogs that have more advanced mitral valve regurgitation that is hemodynamically severe and long-standing enough to have caused radiographic and echocardiographic findings of left atrial and ventricular enlargement that meet clinical trial criteria used to identify dogs that clearly should benefit from initiating pharmacologic treatment to delay the onset of heart failure.
Stage C: dogs have (M)MVD severe enough to cause current or past clinical signs of heart failure. Stage C includes all dogs with (M)MVD that have experienced an episode of clinical heart failure and that are not refractory to standard heart failure treatment. These patients continue to be categorized as Stage C even after improvement or complete resolution of their clinical signs with standard treatment. In exceptional cases that undergo successful surgical mitral valve repair, reclassification to Stage B is warranted.
Stage D: refers to dogs with end-stage (M)MVD, in which clinical signs of heart failure are refractory to standard treatment (defined later in this consensus statement). Such patients require advanced or specialized treatment strategies to remain clinically comfortable with their disease, and at some point, treatment efforts become futile without surgical repair of the valve. As with Stage C, the panel has distinguished between dogs in Stage D that require acute, hospital-based treatment and those that can be managed as outpatients.

Standard treatment is usually recommended as of stage B1 in order to slow progression of the disease, clinical treatment is clearly needed as of stage B2. Management of heart failure is palliative and is aimed at controlling clinical signs related to the presence of oedema and cavity effusion. These are accomplished through reducing preload and/or afterload by diuretics and vasodilators, improving cardiac performance (positive inotropes, positive lusitropes, antiarrhythmics), and using neurohormonal modulators (ACE inhibitors, and potentially β-blockers, aldosterone antagonists, and angiotensin II receptor blockers).

SGLT2 inhibitors were shown to reduce the risk for hospitalisation for heart failure and the risk of new onset of heart failure events in human patients with type II diabetes. A program called EMPEROR was initiated recently to investigate if empagliflozin shows favourable effects in human patients with heart disease independent of diabetes. It was recently announced that the EMPEROR-Reduced Phase III trial as part of the EMPEROR program showed that empagliflozin reduced the risk for the composite endpoint of cardiovascular death or hospitalization due to heart failure in adults with heart failure and reduced ejection fraction, with and without diabetes. However, the pathology of cardiac disease in dogs differs significantly to the pathology observed in humans, where e.g. arteriosclerosis, which is not reported in dogs, is a major concern.

State of the art interventions are based on specific action on unique pathways for symptomatic treatment of secondary conditions induced by cardiac disease, such as positive inotropics (improved contraction), ACE inhibitors (reduce high blood pressure), and diuretics (increased fluid excretion).

Lin Y et al. (J Am Heart Assoc 2021, 10:e019274) discloses that dapagliflozin improves cardiac hemodynamics and mitigates arrhythmogenesis in mitral regurgitation-induced myocardial dysfunction.

Matsumura K et al. (Cardiovascular Ultrasound 2019, 17(1): 26) discloses the effect of SGLT-2 inhibitors on cardiac function and cardiovascular outcome.

Nishinarity R et al. (J Am Heart Assoc 2021, 10:e017483) discloses that canagliflozin suppresses atrial remodeling in a canine atrial fibrillation model.

Santos-Gallego C G et al. (J American College Cardiol 2019, 73(15): 1931-1944) discloses that empagliflozin ameliorates adverse left ventricular remodeling in non-diabetic heart failure by enhancing myocardial energetics.

Silva Custodio Jr J et al. (Heart Failure Reviews 2018, 23(3): 409-418) discloses SGLT-2 inhibition and heart failure current concepts.

US 2011/098240 discloses a pharmaceutical composition comprising a SGLT2 inhibitor in combination with a DPP IV inhibitor, which is suitable in the treatment or prevention of one or more conditions selected from type 1 diabetes mellitus, type 2 diabetes mellitus, impaired glucose tolerance and hyperglycemia.

US 2015/164856 discloses one or more SGLT2 inhibitors or pharmaceutically acceptable forms thereof for use in the treatment and/or prevention of a metabolic disorder in a feline animal, preferably wherein the metabolic disorder is one or more selected from the group consisting of: ketoacidosis, pre-diabetes, diabetes mellitus type 1 or type 2, insulin resistance, obesity, hyperglycemia, impaired glucose tolerance, hyperinsulinemia, dyslipidemia, dysadipokinemia, subclinical inflammation, systemic inflammation, low grade systemic inflammation, hepatic lipidosis, atherosclerosis, inflammation of the pancreas, neuropathy and/or Syndrome X (metabolic syndrome) and/or loss of pancreatic beta cell function and/or wherein the remission of the metabolic disorder, preferably diabetic remission, is achieved and/or maintained.

US 2016/000816 discloses certain SGLT-2 inhibitors for treating and/or preventing oxidative stress, for example in human patients with type 1 or type 2 diabetes, as well as to the use of such SGLT-2 inhibitors in treatment and/or prevention of cardiovascular diseases in human patients, for example type 1 or type 2 diabetes patients.

US 2017/266152 discloses methods for preventing or treating acute or chronic heart failure and for reducing the risk of cardiovascular death, hospitalization for heart failure and other conditions in human patients with preserved or reduced ejection fraction by administering empagliflozin to the patient.

US 2019/076395 discloses the use of certain SGLT-2 inhibitors, such as ertugliflozin or a pharmaceutically acceptable salt or a co-crystal thereof, for treating, reducing the risk of and/or preventing heart failure, myocardial infarction, cardiovascular disease or cardiovascular death in animals without type 2 or type 1 diabetes mellitus, or in animals with pre-diabetes, or in animals with type 2 or type 1 diabetes mellitus or pre-diabetes.

U.S. Pat. No. 10,537,570 discloses the use of pimobendan in a method of reducing the heart size and/or delaying the onset of clinical symptoms in a patient suffering from asymptomatic (occult, preclinical) heart failure, due to mitral valve disease.

WO 2021/092341 discloses sodium-glucose linked transporter inhibitors for the management of chronic kidney disease, hypertension and heart failure in companion animals.

Notwithstanding the disclosures of the above documents, there is a medical need for the prevention and/or treatment of cardiac diseases in a non-human mammal (patient) excluding a feline, in particular a canine (patient).

### SUMMARY OF THE INVENTION

The present invention concerns one or more SGLT-2 inhibitors or pharmaceutically acceptable forms thereof for use in a method of prevention and/or treatment of one or more cardiac diseases in a non-human mammal/non-human mammal patient excluding a feline, in particular a canine/canine patient.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, are also intended to be comprised by the present invention.

In one aspect, the present invention also concerns the one or more SGLT-2 inhibitors or pharmaceutically acceptable forms thereof for the uses as herein disclosed and/or claimed, wherein the one or more cardiac diseases are selected from the group consisting of: heart failure; congestive heart failure; asymptomatic/preclinical/occult heart failure; heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular).

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In one aspect, the present invention also concerns the one or more SGLT-2 inhibitors or pharmaceutically acceptable forms thereof for the uses as herein disclosed and/or claimed, wherein the one or more cardiac diseases are selected from the group consisting of: (myxomatous) mitral valve disease [(M)MVD], clinically overt (myxomatous) mitral valve disease [(M)MVD], asymptomatic/preclinical/occult (myxomatous) mitral valve disease [(M)MVD], dilated cardiomyopathy (DCM), clinically overt dilated cardiomyopathy (DCM), asymptomatic/preclinical/occult dilated cardiomyopathy (DCM).

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In one aspect, the present invention also concerns the one or more SGLT-2 inhibitors or pharmaceutically acceptable forms thereof for the uses as herein disclosed and/or claimed, wherein the one or more cardiac diseases are selected from the group consisting of: (myxomatous) mitral valve disease [(M)MVD], clinically overt (myxomatous) mitral valve disease [(M)MVD], asymptomatic/preclinical/occult (myxomatous) mitral valve disease [(M)MVD].

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In another aspect, the present invention also concerns the one or more SGLT-2 inhibitors or pharmaceutically acceptable forms thereof for the uses as herein disclosed and/or claimed, wherein the one or more SGLT-2 inhibitors are glucopyranosyl-substituted benzene derivatives.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are selected from the group consisting of:
(1) a glucopyranosyl-substituted benzene derivative of the formula (1)

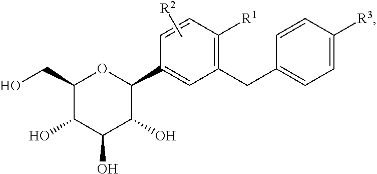

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxycyclohexyl, ethinyl, ethoxy, difluoromethyl, trifluoromethyl, pentafluoroethyl, 2-hydroxylethyl, hydroxymethyl, 3-hydroxy-propyl, 2-hydroxy-2-methyl-prop-1-yl, 3-hydroxy-3-methylbut-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and, most preferably R³ is cyclopropyl, or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2):

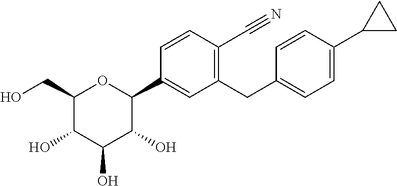 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

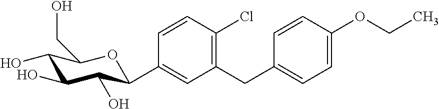

(4) Canagliflozin, represented by formula (4):

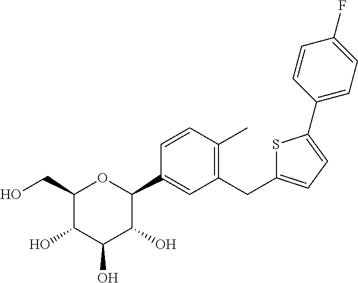 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

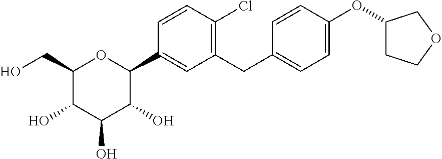

(6) Luseogliflozin, represented by formula (6):

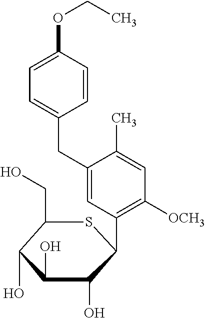

(7) Tofogliflozin represented by formula (7):

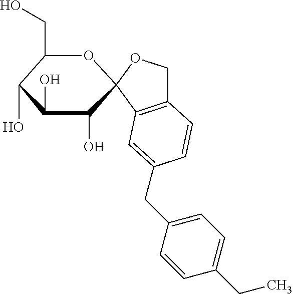

(8) Ipragliflozin, represented by formula (8):

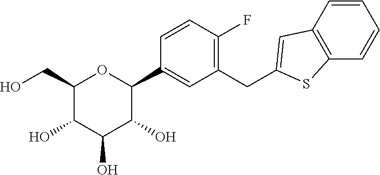 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

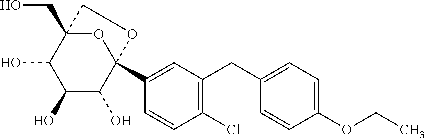 [SMILES: CCOc1ccc(Cc2cc([C@]34OC[C@](CO)(O3)[C@@H](O)[C@H](O)[C@H]4O)ccc2Cl)cc1]

(10) Atigliflozin represented by formula (10):

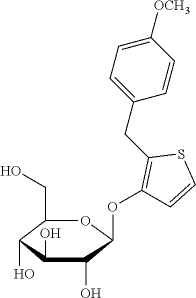

(11) Remogliflozin, represented by formula (11):

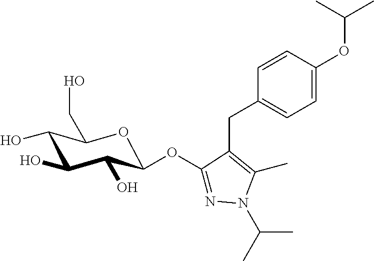

(11A) Remogliflozin etabonate, represented by formula (11A):

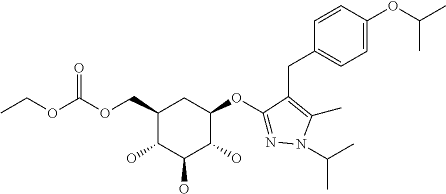 [SMILES: CCOC(=O)OC[C@H]1C[C@@H](Oc2nn(C(C)C)c(C)c2Cc2ccc(OC(C)C)cc2)[C@H](O)[C@@H](O)[C@@H]1O]

(12) a thiophene derivative of the formula (12)

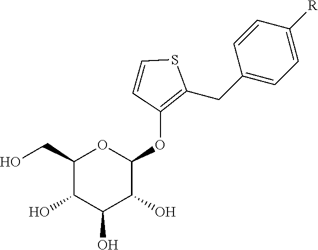

wherein R denotes methoxy or trifluoromethoxy;
(13) 1-(β-D-glucopyranosyl)-4-methyl-3-[5-(4-fluorophenyl)-2-thienylmethyl]benzene, represented by formula (13);

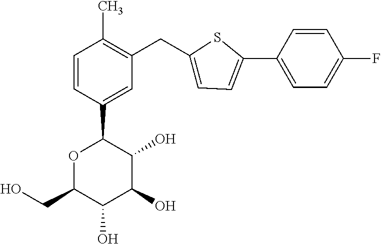 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

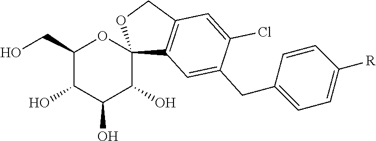

wherein R denotes methoxy, trifluoromethoxy, ethoxy, ethyl, isopropyl or tert. butyl;
(15) a pyrazole-O-glucoside derivative of the formula (15)

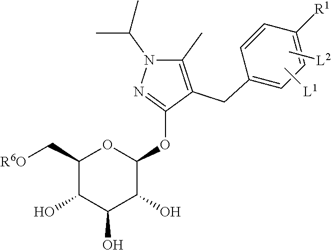

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

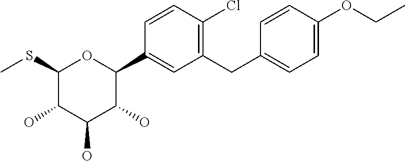 [SMILES: CCOc1ccc(Cc2cc([C@@H]3O[C@H](SC)[C@@H](O)[C@H](O)[C@H]3O)ccc2Cl)cc1]

(17) Sergliflozin represented by formula (17):

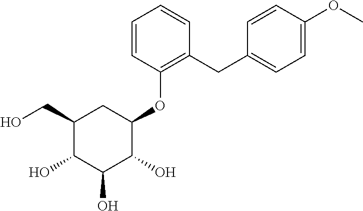 [SMILES: COc1ccc(Cc2ccccc2O[C@@H]2C[C@H](CO)[C@@H](O)[C@H](O)[C@H]2O)cc1]

(18) a compound represented by formula (18):

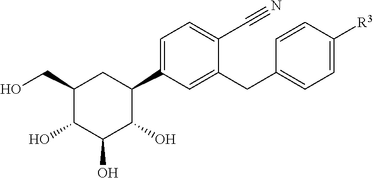

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
   or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

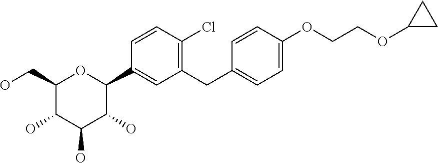 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

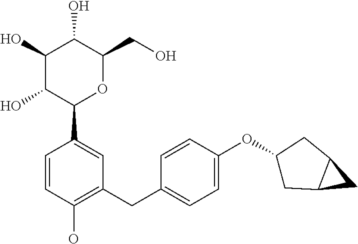 [SMILES: OC[C@H]1O[C@@H](c2ccc(O)c(Cc3ccc(O[C@H]4C[C@@H]5C[C@@H]5C4)cc3)c2)[C@H](O)[C@@H](O)[C@@H]1O]

(21) Rongliflozin, represented by formula (21):

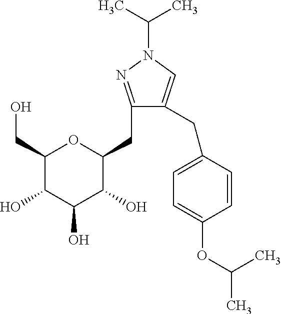 [SMILES: CC(C)Oc1ccc(Cc2cn(C(C)C)nc2C[C@@H]2O[C@H](CO)[C@@H](O)[C@H](O)[C@H]2O)cc1]

(22) Wanpagliflozin.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the pharmaceutically acceptable form thereof is a crystalline complex between the one or more SGLT2 inhibitors and one or more amino acids, preferably proline, more preferably L-proline; and most preferably is co-crystal of the one or more SGLT2 inhibitors, L-proline and crystalline water.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the non-human mammal/non-human mammal patient excluding a feline, in particular a canine/canine patient, is a patient in need of such prevention and/or treatment; and preferably is a dog in need of such prevention and/or treatment, more preferably a non-diabetic dog in need of such prevention and/or treatment.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT2 inhibitors are administered orally, parenterally, intravenously, subcutaneously or intramuscularly, preferably orally.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT2 inhibitors are to be administered at a dose of 0.01 mg/kg bodyweight to 10 mg/kg bodyweight per day, preferably at a dose of 0.01 mg/kg bodyweight to 5 mg/kg bodyweight per day, more preferably at a dose of 0.01 mg/kg bodyweight to 4 mg/kg bodyweight per day, even more preferably at a dose of 0.01 mg/kg bodyweight to 3 mg/kg bodyweight per day, even more preferably at a dose of 0.01 mg/kg bodyweight to 2 mg/kg bodyweight per day, even more preferably at a dose of 0.01 mg/kg bodyweight to 1 mg/kg bodyweight per day, even more preferably at a dose of 0.01 mg/kg bodyweight to 0.5 mg/kg bodyweight per day, most preferably at a dose of 0.01 mg/kg bodyweight to 0.3 mg/kg bodyweight per day.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein such one or more SGLT2 inhibitors or pharmaceutically acceptable forms thereof is to be administered once or twice per day. A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT2 inhibitors is velagliflozin, which is to be administered as single SGLT-2 inhibitor, preferably orally, more preferably once or twice per day at a dose of 0.01 mg/kg bodyweight to 1 mg/kg bodyweight, even more preferably at a dose of 0.01 mg/kg bodyweight to 0.5 mg/kg bodyweight, even more preferably at a dose of 0.01 mg/kg bodyweight to 0.3 mg/kg bodyweight, most preferably once daily at a dose of 0.05 mg/kg bodyweight.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein velagliflozin as single SGLT-2 inhibitor is to be orally administered once daily at a dose of 0.01 mg/kg bodyweight to 0.3 mg/kg bodyweight, preferably once daily at a dose of 0.05 mg/kg bodyweight.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT2 inhibitors are to be administered before, after or concomitantly with administering one or more other active pharmaceutical ingredients, preferably selected from the group consisting of: another SGLT-2 inhibitor or a pharmaceutically acceptable form thereof, one or more diuretics, such as furosemide, torasemide or spironolactone; one or more beta-blockers, such as atenolol or propranolol; one or more calcium-channel blockers, such as amlodipine and diltiazem; one or more ACE inhibitors, such as benazepril, ramipril or enalapril; one or more angiotensin receptors blockers, such as telmisartan; one or more antiarrhythmic agents, such as flecainide; one or more platelet agglutination inhibitors, such as clopidogrel; one or more nonsteroidal anti-inflammatory drugs (NSAIDs), such as aspirin; one or more anticoagulants, such as Coumarins (vitamin K antagonists), (low molecular weight) heparin, synthetic pentasaccharide inhibitors of factor Xa, as well as direct factor Xa inhibitors and/or direct thrombin inhibitors; and/or one or more calcium-channel sensitizers and/or positive inotropes, such as pimobendan and/or digitalis alkaloids.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the preventive and/or therapeutic effect is characterized by one or more of the following clinical and/or biochemical parameters:
improved cardiometabolic efficiency, characterized by an increased ratio of [cardiac output/metabolic substrate consumed] and/or characterized by an increased ratio of [cardiac output/oxygen consumed];
increase of the production of ketone bodies in the liver, characterized by increased plasma levels of 3-hydroxybutyric acid and/or the corresponding acylcarnitines, i.e. hydroxybutyrylcarnitine, and increased plasma levels of one or more of the branched-chain amino acids (valine, leucine and isoleucine);
improved cardiac function by achieved reduced pre- and/or afterload, improved arterial wall structure function;
improved echocardiographic parameters, such as decreased LA (Left atrium dimension measured as right parasternal short-axis), LA/Ao (left atrium to aorta ratio; Ao=Aortic root diameter), IVSd (interventricular septal end diastolic dimension, i.e. the thickness of the interventricular septum), and/or LAD (Left atrium measured as right parasternal long-axis), and improved cardiac biomarkers, such as decreased NT-proBNP (N-terminal prohormone of brain natriuretic peptide) and/or decreased cTnI (cardiac Troponin I) and/or increased erythropoietin concentration, as well as improved heart murmur;
delayed onset of different phenotypes of cardiac diseases, such as (M)MVD and/or DCM, preferably at least by 1, 2, 3, 4, 5, 6, 7, 8, 9, 10, 11, 12, 13, 14, 15 or more months, or even stopped progression of different phenotypes of cardiac diseases, such as (M)MVD and/or DCM;
longer time of survival, preferably at least by 1, 2, 3, 4, 5, 6, 7, 8, 9, 10, 11, 12, 13, 14, 15 or more months, and/or delay of next episode of heart failure, preferably at least by 1, 2, 3, 4, 5, 6, 7, 8, 9, 10, 11, 12, 13, 14, 15 or more months, and/or lower level of cardiac mortality and/or morbidity;
improved clinical signs, such as reduced e.g. breathlessness or dyspnea, cough, depression, exercise intolerance, inappetence, syncope, abdominal distention and/or polydipsia;
prolongation of time to event (e.g. heart failure, cardiac death, onset of clinical signs, need for additional concomitant medication, increase in dose of concomitant therapy—diuretics);
higher quality of life.

A corresponding method of preventing and/or treating one or more cardiac diseases in a non-human mammal/non-human mammal patient excluding a feline, in particular a canine/canine patient, comprising administering one or more SGLT-2 inhibitors to such non-human mammal/non-human mammal patient excluding a feline, in particular a canine/canine patient, as herein disclosed and/or claimed, as well as the corresponding use of one or more SGLT-2 inhibitors for the preparation of a medicament for the prevention and/or treatment of one or more cardiac diseases in a non-human mammal/non-human mammal patient excluding a feline, in particular a canine/canine patient, as herein disclosed and/or claimed, are also intended to be comprised by the present invention.

The present invention further concerns a pharmaceutical composition comprising one or more SGLT2 inhibitors or pharmaceutically acceptable forms thereof as herein disclosed and/or claimed for the uses/methods as herein disclosed and/or claimed.

The advantages according to the present invention are one or more of the following:
improved cardiometabolic efficiency, characterized by an increased ratio of [cardiac output/metabolic substrate consumed] and/or characterized by an increased ratio of [cardiac output/oxygen consumed];
increase of the production of ketone bodies in the liver, characterized by increased plasma levels of 3-hydroxybutyric acid and/or the corresponding acylcarnitines, i.e. hydroxybutyrylcarnitine, and increased plasma levels of one or more of the branched-chain amino acids (valine, leucine and isoleucine);
improved cardiac function by achieved reduced pre- and/or afterload, improved arterial wall structure function;
improved echocardiographic parameters, such as decreased LA (Left atrium dimension measured as right parasternal short-axis), LA/Ao (left atrium to aorta ratio; Ao=Aortic root diameter), IVSd (interventricular septal end diastolic dimension, i.e. the thickness of the interventricular septum), and/or LAD (Left atrium measured as right parasternal long-axis), and improved cardiac biomarkers, such as decreased NT-proBNP (N-terminal prohormone of brain natriuretic peptide) and/or decreased cTnI (cardiac Troponin I) and/or increased erythropoietin concentration, as well as improved heart murmur;
delayed onset of different phenotypes of cardiac diseases, such as (M)MVD and/or DCM, preferably at least by 1, 2, 3, 4, 5, 6, 7, 8, 9, 10, 11, 12, 13, 14, 15 or more months, or even stopped progression of different phenotypes of cardiac diseases, such as (M)MVD and/or DCM;
longer time of survival, preferably at least by 1, 2, 3, 4, 5, 6, 7, 8, 9, 10, 11, 12, 13, 14, 15 or more months, and/or delay of next episode of heart failure, preferably at least by 1, 2, 3, 4, 5, 6, 7, 8, 9, 10, 11, 12, 13, 14, 15 or more months, and/or lower level of cardiac mortality and/or morbidity;
improved clinical signs, such as reduced e.g. breathlessness or dyspnea, cough, depression, exercise intolerance, inappetence, syncope, abdominal distention and/or polydipsia;
prolongation of time to event (e.g. heart failure, cardiac death, onset of clinical signs, need for additional concomitant medication, increase in dose of concomitant therapy—diuretics);
higher quality of life of dogs with cardiac diseases, such as (M)MVD and/or DCM.

### DETAILED DESCRIPTION OF THE INVENTION

Before the embodiments of the present invention are described in further detail, it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

In the course of the present invention, the term “heart disease” is synonymous with “cardiac disease” and refers to any disorder and deformities of the heart itself, which affect the heart's structure and function. There are many types of heart disease that affect different parts of the organ and occur in different ways including congenital heart diseases (e.g. septal defects, obstruction defects), arrhythmias (e.g. tachycardia, bradycardia and fibrillation) and cardiomyopathies.

In the course of the present invention, the term “heart failure”, also known as congestive heart failure and congestive cardiac failure, refers to the pathophysiological process in which the heart cannot pump sufficiently to maintain the blood flow through the body to meet the metabolic requirements (oxygen and substrates) of peripheral tissues and organs. It can also be defined as a complex clinical syndrome that is based on abnormal structure or function of the heart and which is characterized by symptoms like exercise intolerance, dyspnoea, fatigue, fluid retention and reduced longevity. It can be divided into systolic failure, where the ejection of blood out of the heart in the systole is affected, and diastolic failure, where the heart is not able to receive enough blood in the ventricular cavities at low pressure during diastole. It is mostly a chronic disease due to a chronic work overload of the heart or developed after an acute hemodynamic stress due to fluid overload, a valvular dysfunction or a myocardial infarction.

In the course of the present invention, the term “(myxomatous) mitral valve disease” [(M)MVD] refers to the most common cardiovascular condition in dogs and the most frequent cause of (congestive) heart failure, affecting primally small breed dogs over 5 years of age. The pathophysiology of (myxomatous) mitral valve disease is characterized by the progressive dilation of the left ventricle and the left atrium resulting from degenerative changes and an insufficiency of the mitral valve. The valve defect leads to a blood back-flow and reduced ejection fraction and represents an additional effort to the heart causing an enlargement of the left ventricle that if untreated, weakens, leading to congestive heart failure (CHF).

In the course of the present invention, the term “dilated cardiomyopathy” (DCM) refers to the second most common cardiovascular condition in dogs, affecting primally larger dogs of all ages with an overall incidence of 8%. DCM is the disease of the cardiomyocytes, presenting itself by an enlargement of the left ventricle and left atrium or an enlargement of all the heart chambers and often the muscular walls of the heart are much thinner than normal. As a result, the ventricle's pump capacity becomes reduced and blood flow is impaired, leading to a congestion in the blood flow. As the disease progresses, it causes congestive heart failure (CHF).

In the course of the present invention, the term “asymptomatic (occult, preclinical) (myxomatous) mitral valve disease [(M)MVD]” relates to any contractile disorder or disease of the heart which is due to/secondary to (M)MVD—however, yet without any clinical symptoms of (congestive) heart failure. In particular, it relates to heart failure due to (M)MVD of ISACHC Class I (Class IA and/or Class IB), NYHA Class I and ACVIM stage B2.

In the course of the present invention, the term “asymptomatic (occult, preclinical) (myxomatous) dilated cardiomyopathy (DCM)” relates to any contractile disorder or disease of the heart which is due to/secondary to DCM—however, yet without any clinical symptoms of (congestive) heart failure. In particular, it relates to heart failure due to DCM of ISACHC Class I.

In the course of the present invention, the term “canine animal” or “canine” refers to any member of the canidae family (i.e. a canid). It may thus belong either to the subfamily canidae or the subfamily caninae. The term canine animal encompasses the term dog, e.g., a domestic dog. The term domestic dog encompasses the terms Canis familiaris or Canis lupus familiaris. Most preferably, the canine animal or canine is a dog, in particular a domestic dog.

In a preferred embodiment, the “non-human mammal” is selected from the group consisting of: bovine, canine, caprine, equine, lagomorphs, ovine, porcine, rodent; more preferably is selected from the group consisting of: cattle, cow, dog, goat, horse, pony, donkey, sheep, pig, rabbit, rat, mouse; even more preferably selected from the group consisting of: canine; most preferably selected from the group consisting of: dog.

SGLT-2 inhibitors for use according to the invention include, but are not limited to, glucopyranosyl-substituted benzene derivatives, for example as described in WO 01/27128, WO 03/099836, WO 2005/092877, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, WO 2008/055940, WO 2009/022020 or WO 2009/022008.

Moreover, the one or more SGLT-2 inhibitors for use according to the invention may be selected from the group consisting of the following compounds or pharmaceutically acceptable forms thereof:
(1) a glucopyranosyl-substituted benzene derivative of the formula (1)

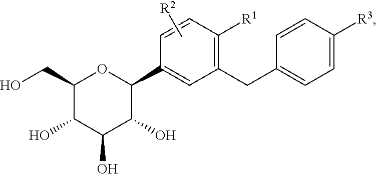

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxycyclohexyl, ethinyl, ethoxy, difluoromethyl, trifluoromethyl, pentafluoroethyl, 2-hydroxylethyl, hydroxymethyl, 3-hydroxy-propyl, 2-hydroxy-2-methyl-prop-1-yl, 3-hydroxy-3-methylbut-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl, or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2):

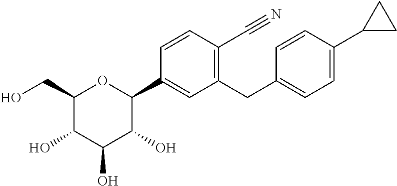 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

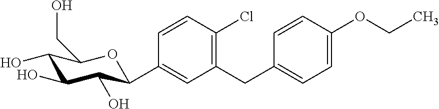

(4) Canagliflozin, represented by formula (4):

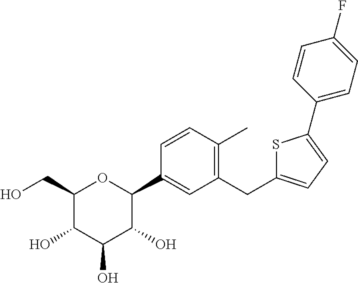 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

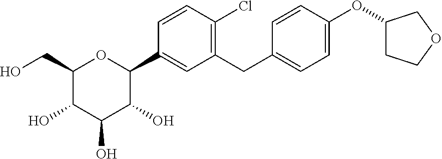

(6) Luseogliflozin, represented by formula (6):

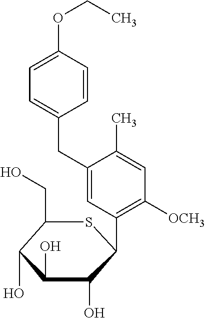

(7) Tofogliflozin, represented by formula (7):

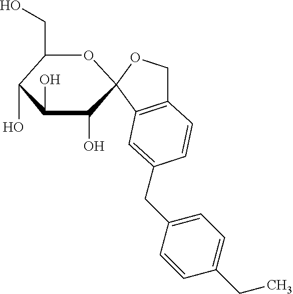

(8) Ipragliflozin, represented by formula (8):

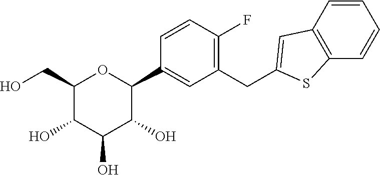 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

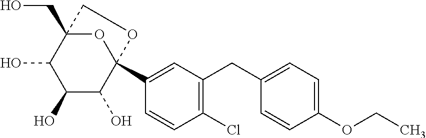 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by formula (10):

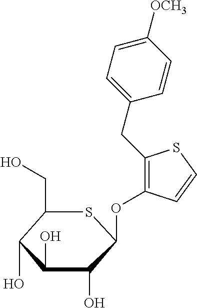

(11) Remogliflozin, represented by formula (11):

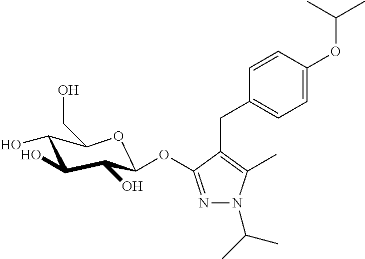

(11A) Remogliflozin etabonate, represented by formula (11A):

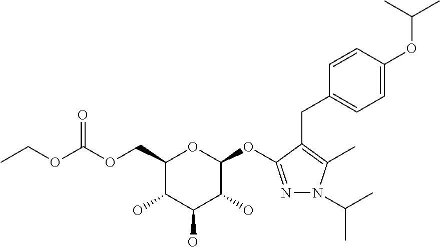 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

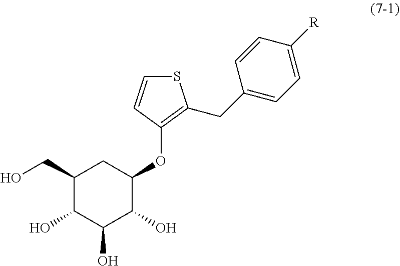

wherein R denotes methoxy or trifluoromethoxy;
(13) 1-(β-D-glucopyranosyl)-4-methyl-3-[5-(4-fluorophenyl)-2-thienylmethyl]benzene, represented by formula (13);

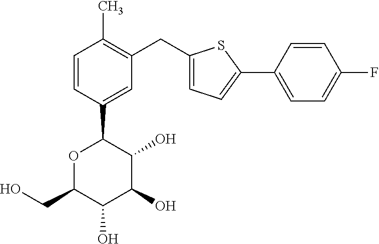 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

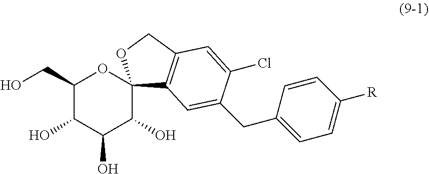

wherein R denotes methoxy, trifluoromethoxy, ethoxy, ethyl, isopropyl or tert. butyl;
(15) a pyrazole-O-glucoside derivative of the formula (15)

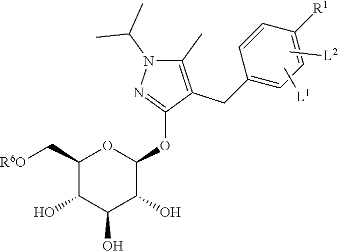

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

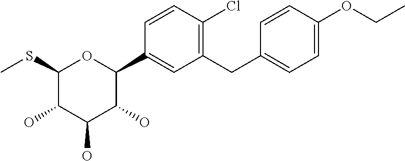 [SMILES: CCOc1ccc(Cc2cc([C@@H]3O[C@H](SC)[C@@H](O)[C@H](O)[C@H]3O)ccc2Cl)cc1]

(17) Sergliflozin represented by formula (17):

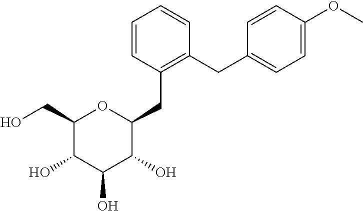 [SMILES: COc1ccc(Cc2ccccc2C[C@@H]2O[C@H](CO)[C@@H](O)[C@H](O)[C@H]2O)cc1]

(18) a compound represented by formula (18):

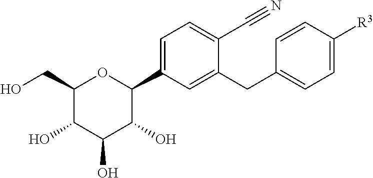

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

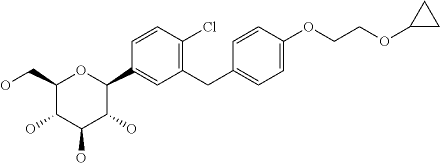 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

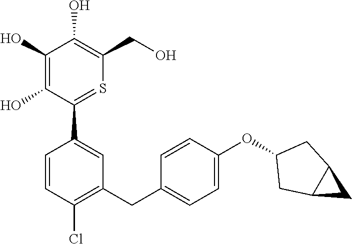

(21) Rongliflozin, represented by formula (21):

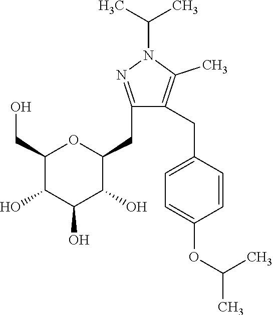 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin.

The term “velagliflozin” as employed herein refers to velagliflozin of the above structure as well as pharmaceutically acceptable forms thereof, including hydrates and solvates thereof, and crystalline forms thereof. The compound, methods of its synthesis and co-crystals thereof are described in WO 2007/128749, WO 2014/016381 and WO 2019/121509 for example.

The term “dapagliflozin” as employed herein refers to dapagliflozin of the above structure as well as pharmaceutically acceptable forms thereof, including hydrates and solvates thereof, and crystalline forms thereof. The compound and methods of its synthesis are described in WO 03/099836 for example. Preferred hydrates, solvates and crystalline forms are described in the patent applications WO 2008/116179 and WO 2008/002824 for example.

The term “canagliflozin” as employed herein refers to canagliflozin of the above structure as well as pharmaceutically acceptable forms thereof, including hydrates and solvates thereof, and crystalline forms thereof. The compound and methods of its synthesis are described in WO 2005/012326 and WO 2009/035969 for example. Preferred hydrates, solvates and crystalline forms are described in the patent application WO 2008/069327 for example.

The term “empagliflozin” as employed herein refers to empagliflozin of the above structure as well as pharmaceutically acceptable forms thereof, including hydrates and solvates thereof, and crystalline forms thereof. The compound and methods of its synthesis are described in WO 2005/092877, WO 2006/120208 and WO 2011/039108 for example. A preferred crystalline form is described in the patent applications WO 2006/117359 and WO 2011/039107 for example.

The term “atigliflozin” as employed herein refers to atigliflozin of the above structure as well as pharmaceutically acceptable forms thereof, including hydrates and solvates thereof, and crystalline forms thereof. The compound and methods of its synthesis are described in WO 2004/007517 for example.

The term “ipragliflozin” as employed herein refers to ipragliflozin of the above structure as well as pharmaceutically acceptable forms thereof, including hydrates and solvates thereof, and crystalline forms thereof.

The compound and methods of its synthesis are described in WO 2004/080990, WO 2005/012326 and WO 2007/114475 for example.

The term “tofogliflozin” as employed herein refers to tofogliflozin of the above structure as well as pharmaceutically acceptable forms thereof, including hydrates and solvates thereof, and crystalline forms thereof. The compound and methods of its synthesis are described in WO 2007/140191 and WO 2008/013280 for example.

The term “luseogliflozin” as employed herein refers to luseogliflozin of the above structure as well as pharmaceutically acceptable forms thereof, including hydrates and solvates thereof, and crystalline forms thereof.

The term “ertugliflozin” as employed herein refers to ertugliflozin of the above structure as well as pharmaceutically acceptable forms thereof, including hydrates and solvates thereof, and crystalline forms thereof. The compound is described for example in WO 2010/023594.

The term “remogliflozin” as employed herein refers to remogliflozin of the above structure as well as pharmaceutically acceptable forms thereof, including prodrugs of remogliflozin, in particular remogliflozin etabonate, including hydrates and solvates thereof, and crystalline forms thereof. Methods of its synthesis are described in the patent applications EP 1 213 296 and EP 1 354 888 for example.

The term “sergliflozin” as employed herein refers to sergliflozin of the above structure as well as pharmaceutically acceptable forms thereof, including prodrugs of sergliflozin, in particular sergliflozin etabonate, including hydrates and solvates thereof, and crystalline forms thereof. Methods for its manufacture are described in the patent applications EP 1 344 780 and EP 1 489 089 for example.

The compound of formula (16) above, i.e. sotagliflozin, and its manufacture are described for example in WO 2008/042688 or WO 2009/014970.

Preferred SGLT-2 inhibitors are glucopyranosyl-substituted benzene derivatives. Optionally, one or more hydroxyl groups of the glucopyranosyl group in such one or more SGLT-2 inhibitors may be acylated with groups selected from (C₁₋₁₈-alkyl)carbonyl, (C₁₋₁₈-alkyl)oxycarbonyl, phenylcarbonyl and phenyl-(C₁₋₃-alkyl)carbonyl.

More preferred are glucopyranosyl-substituted benzonitrile derivatives of formula (1) as disclosed herein above. Yet more preferred are glucopyranosyl-substituted benzonitrile derivatives of formula (18):

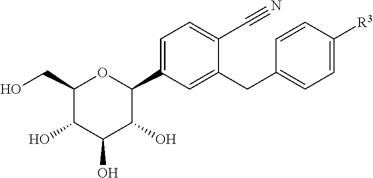

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxypropyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl, or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Preferably, such SGLT-2 inhibitor is velaglifozin as shown in formula (2). Optionally, one or more hydroxyl groups of the β-D-glucopyranosyl group of velagliflozin may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Thus, in a preferred embodiment, the at least one SGLT-2 inhibitor according to the present invention is a glucopyranosyl-substituted benzene derivative SGLT-2 inhibitor, preferably a SGLT-2 inhibitor of formula (1), more preferably of formula (18), or yet more preferably of formula (2), i.e. velagliflozin, in each case as defined herein above.

Herein, references to SGLT-2 inhibitors and/or their use according to the invention encompass pharmaceutically acceptable forms of the SGLT-2 inhibitors, unless otherwise stated.

According to the invention, any pharmaceutically acceptable form of the SGLT-2 inhibitor, e.g. of formula (1), preferably formula (18), more preferably formula (2), may be used. E.g. a crystalline form may be used. Prodrug forms are also encompassed by the present invention.

Prodrug forms may include, e.g., esters and/or hydrates. The term “prodrug” is also meant to include any covalently bonded carrier, which releases the active compound of the invention in vivo when the prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention.

Crystalline forms for use according to the invention include a complex of an SGLT-2 inhibitor with one or more amino acids (see e.g. WO 2014/016381)—so-called co-crystals. An amino acid for such use may be a natural amino acid. The amino acid may be a proteogenic amino acid (including L-hydroxyproline), or a non-proteogenic amino acid. The amino acid may be a D- or an L-amino acid. In some preferred embodiments, the amino acid is proline (L-proline and/or D-proline, preferably L-proline). E.g., a crystalline complex/co-crystal of velagliflozin with proline (e.g. L-proline) and crystalline water is preferred.

Thus, herein is disclosed a crystalline complex/co-crystal between one or more natural amino acids and an SGLT-2 inhibitor, e.g., a crystalline complex/co-crystal between one or more natural amino acids and a glucopyranosyl-substituted benzene derivative SGLT-2 inhibitor, preferably a SGLT-2 inhibitor of formula (1), more preferably of formula (18) or yet more preferably of formula (2) (velagliflozin).

A certain pharmaceutical activity is the basic prerequisite to be fulfilled by a pharmaceutically active agent before it is approved as a medicament on the market. However, there are a variety of additional requirements a pharmaceutically active agent has to comply with. These requirements are based on various parameters, which are connected with the nature of the active substance itself. Without being restrictive, examples of these parameters are the stability of the active agent under various environmental conditions, its stability during production of the pharmaceutical formulation and the stability of the active agent in the final medicament compositions. The pharmaceutically active substance used for preparing the pharmaceutical compositions should be as pure as possible and its stability in long-term storage must be guaranteed under various environmental conditions. This is essential to prevent the use of pharmaceutical compositions, which contain, in addition to the actual active substance, breakdown products thereof, for example. In such cases, the content of active substance in the medicament might be less than that specified.

Uniform distribution of the medicament in the formulation is a critical factor, particularly when the medicament has to be given in low doses. To ensure uniform distribution, the particle size of the active substance can be reduced to a suitable level, e.g. by grinding. Since breakdown of the pharmaceutically active substance as a side effect of the grinding (or micronizing) has to be avoided as far as possible, in spite of the hard conditions required during the process, it is essential that the active substance should be highly stable throughout the grinding process. Only if the active substance is sufficiently stable during the grinding process it is possible to produce a homogeneous pharmaceutical formulation, which always contains the specified amount of active substance in a reproducible manner.

Another problem, which may arise in the grinding process for preparing the desired pharmaceutical formulation, is the input of energy caused by this process and the stress on the surface of the crystals. This may in certain circumstances lead to polymorphous changes, to amorphization or to a change in the crystal lattice. Since the pharmaceutical quality of a pharmaceutical formulation requires that the active substance should always have the same crystalline morphology, the stability and properties of the crystalline active substance are subject to stringent requirements from this point of view as well.

The stability of a pharmaceutically active substance is also important in pharmaceutical compositions for determining the shelf life of the particular medicament; the shelf life is the length of time during which the medicament can be administered without any risk. High stability of a medicament in the abovementioned pharmaceutical compositions under various storage conditions is therefore an additional advantage for both the patient and the manufacturer.

The absorption of moisture reduces the content of pharmaceutically active substance because of the increased weight caused by the uptake of water. Pharmaceutical compositions with a tendency to absorb moisture have to be protected from moisture during storage, e.g. by the addition of suitable drying agents or by storing the drug in an environment where it is protected from moisture. Preferably, therefore, a pharmaceutically active substance should be at best slightly hygroscopic.

Furthermore, the availability of a well-defined crystalline form allows the purification of the drug substance by recrystallization.

Apart from the requirements indicated above, it should be generally borne in mind that any change to the solid state of a pharmaceutical composition, which is capable of improving its physical and chemical stability, gives a significant advantage over less stable forms of the same medicament.

A crystalline complex/co-crystal between a natural amino acid and an SGLT-2 inhibitor (e.g. a glucopyranosyl-substituted benzene derivative or a SGLT-2 inhibitor of formula (1), or formula (18) or, particularly, of formula (2), i.e. velagliflozin) fulfills important requirements mentioned hereinbefore.

SGLT-2 inhibitors for use according to the invention may be prepared as pharmaceutical compositions. They may be prepared as solid or as liquid formulations. In either case, they are preferably prepared for oral administration, preferably in liquid form for oral administration (see e.g. WO 2017/032799). The SGLT-2 inhibitors may, however, also be prepared, e.g., for parenteral administration. Solid formulations include tablets, granular forms, and other solid forms such as suppositories. Among solid formulations, tablets and granular forms are preferred.

Pharmaceutical compositions within the meaning of the present invention may comprise an SGLT-2 inhibitor according to the present invention and one or more excipients. Any excipient that allows for, or supports, the intended medical effect may be used. Such excipients are available to the skilled person. Useful excipients are for example anti-adherents (used to reduce the adhesion between the powder (granules) and the punch faces and thus prevent sticking to tablet punches), binders (solution binders or dry binders that hold the ingredients together), coatings (to protect tablet ingredients from deterioration by moisture in the air and make large or unpleasant-tasting tablets easier to swallow), disintegrants (to allow the tablet to break upon dilution), fillers, diluents, flavours, colours, glidants (flow regulators—to promote powder flow by reducing interparticle friction and cohesion), lubricants (to prevent ingredients from clumping together and from sticking to the tablet punches or capsule filling machine), preservatives, sorbents, sweeteners etc.

Formulations according to the invention, e.g. solid formulations, may comprise carriers and/or disintegrants selected from the group of sugars and sugar alcohols, e.g. mannitol, lactose, starch, cellulose, microcrystalline cellulose and cellulose derivatives, e.g. methylcellulose, and the like.

Manufacturing procedures for formulations suitable for canines are known to the person skilled in the art, and for solid formulations comprise, e.g., direct compression, dry granulation and wet granulation. In the direct compression process, the active ingredient and all other excipients are placed together in a compression apparatus that is directly applied to press tablets out of this material. The resulting tablets can optionally be coated afterwards in order to protect them physically and/or chemically, e.g. by a material known from the state of the art.

A unit for administration, e.g. a single liquid dose or a unit of a solid formulation, e.g. a tablet, may comprise 0.1 mg to 10 mg, or e.g. 0.3 mg to 1 mg, 1 mg to 3 mg, 3 mg to 10 mg; or 5 to 2500 mg, or e.g. 5 to 2000 mg, 5 mg to 1500 mg, 10 mg to 1500 mg, 10 mg to 1000 mg, or 10-500 mg of an SGLT-2 inhibitor for use according to the invention. As the skilled person would understand, the content of the SGLT-2 inhibitor in a solid formulation, or any formulation as disclosed herein for administration to a canine animal, may be increased or decreased as appropriate in proportion to the body weight of the canine animal to be treated.

In one embodiment, a pharmaceutical composition for use according to the invention is designed for oral or parenteral administration, preferably for oral administration. Especially the oral administration is ameliorated by excipients, which modify the smell and/or haptic properties of the pharmaceutical composition for the intended patient, e.g. as described.

When the SGLT-2 inhibitor for use according to the invention is formulated for oral administration, it is preferred that excipients confer properties, e.g. palatability and/or chewability that render the formulation suitable for administration to a canine animal.

Also preferred are liquid formulations. Liquid formulations may be, e.g., solutions, syrups or suspensions. They may be administered directly to the canines or may be mixed with the food and/or drink (e.g. drinking water, or the like) of the canine animal. One advantage of a liquid formulation (similar to a formulation in granular form), is that such a dosage form allows precise dosing. For example, the SGLT-2 inhibitor may be dosed precisely in proportion to the body mass of a canine animal. Typical compositions of liquid formulations are known to the person skilled in the art.

A practitioner skilled in the art can determine suitable doses for the uses of the present invention. Preferred units dosing units include mg/kg bodyweight, i.e. mg SGLT-2 inhibitor per body mass of the non-human mammal excluding feline, in particular canine animal. An SGLT-2 inhibitor of the invention may, e.g., be administered in doses of 0.01-10 mg/kg bodyweight per day, e.g. 0.01-5 mg/kg bodyweight per day, e.g. 0.01-4 mg/kg bodyweight per day, e.g. 0.01-3 mg/kg bodyweight per day, e.g. 0.01-2 mg/kg bodyweight per day, e.g. 0.01-1.5 mg/kg bodyweight per day, e.g., 0.01-1 mg/kg bodyweight per day, e.g. 0.01-0.75 mg/kg bodyweight per day, e.g. 0.01-0.5 mg/kg bodyweight per day, e.g. 0.01-0.4 mg/kg bodyweight per day; or 0.1 to 3.0 mg/kg bodyweight per day, preferably from 0.2 to 2.0 mg/kg bodyweight per day, more preferably from 0.1 to 1 mg/kg bodyweight per day or from 0.5 to 1 mg/kg bodyweight per day. In another preferred embodiment, the dose is 0.01-1 mg/kg bodyweight per day, preferably 0.01-0.5 mg/kg bodyweight per day, more preferably 0.02-0.4 mg/kg bodyweight per day, e.g. 0.03-0.3 mg/kg bodyweight per day.

A practitioner skilled in the art is able to prepare an SGLT-2 inhibitor of the invention for administration according to a desired dose.

### EXAMPLES

The following examples serve to further illustrate the present invention; but the same should not be construed as a limitation of the scope of the invention disclosed herein.

### Example 1 Exploratory Clinical Field Study “DCM”—Outline

Client-owned dog patients (older than 1 year) diagnosed with DCM via physical and echocardiography (modified NYHA class 2 and 3) are treated orally and once daily with a velagliflozin dosage of 0.3 mg/kg bodyweight. During the study period the body weight, body condition score, blood pressure and the cardiovascular system are examined on a regular basis during the visits at the site by the investigator. Additionally, thoracic radiographs (in the right lateral and dorso ventral view), echocardiography (IVSd, LA diameter, Ao diameter, LVIDd, LVWd, LVWs, IVSd, LVPWd, EDV, ESV, EF, % FS, presence of effusion) and electrocardiography are performed. In order to obtain an overview as complete as possible regular blood tests include a complete hematology (white blood cells (WBC), WBC differential, red blood cells, hemoglobin, hematocrit, Heinz bodies, platelet count), a biochemistry panel (total protein, Albumin, Globulin, Alkaline phosphatase (ALP), Alanine transaminase (ALT), Aspartate transaminase (AST), Total bilirubin, Creatinine, Blood urea nitrogen or urea (BUN), Calcium, Sodium, Potassium, Chloride, Phosphorus, Glucose, Cholesterol, Triglycerides, Fructosamine), the measurement of total T4, ketone bodies and cardiac biomarkers (Plasma NT-pro BNP, Cardiac troponin I).

Variables of interest are the number of events defined as cardiac death, cardiac related euthanasia and cardiac disease stage progression and the time to event (survival time of the dog patients). The results of the exploratory clinical field study show a clinically relevant prolongation of survival time and the time to event (event was defined as overall death, cardiovascular death or hospitalization for heart failure). Additionally, clinical parameters (e.g. appetite, activity level and breathing) improve significantly.

### Example 2 Exploratory Laboratory Trial “MVD”—Outline

Laboratory animals (older than 1 year) diagnosed with mitral-valve disease via physical and echocardiography (according to ACVIM guidelines—stage B2 and C) are treated orally and once daily with a velagliflozin dosage of 0.3 mg/kg bodyweight. During the study period the body weight, body condition score, blood pressure and the cardiovascular system are examined on a regular basis during the visits at the site by the investigator. Additionally, thoracic radiographs (in the right lateral and dorso ventral view), echocardiography (IVSd, LA diameter, Ao diameter, LVIDd, LVWd, LVWs, IVSd, LVPWd, EDV, ESV, EF, % FS, presence of effusion) and electrocardiography are performed. In order to obtain an overview as complete as possible regular blood tests include a complete hematology (white blood cells (WBC), WBC differential, red blood cells, hemoglobin, hematocrit, Heinz bodies, platelet count), a biochemistry panel (total protein, Albumin, Globulin, Alkaline phosphatase (ALP), Alanine transaminase (ALT), Aspartate transaminase (AST), Total bilirubin, Creatinine, Blood urea nitrogen or urea (BUN), Calcium, Sodium, Potassium, Chloride, Phosphorus, Glucose, Cholesterol, Triglycerides, Fructosamine), the measurement of total T4, ketone bodies and cardiac biomarkers (Plasma NT-pro BNP, Cardiac troponin I).

Variables of interest are the number of events defined as cardiac death, cardiac related euthanasia and cardiac disease stage progression and the time to event (survival time of the dog patients). The results of the exploratory laboratory trial show a clinically relevant prolongation of survival time and the time to event (event was defined as death or hospitalization for heart failure, pulmonary oedema). Additionally, clinical parameters (e.g. appetite, activity level and breathing) improve significantly.

### Example 3 Exploratory Clinical Field Study in Dogs with Preclinical (Occult) or Clinical (Overt) Dilated Cardiomyopathy (DCM)

The clinical study was conducted at two small animal clinics. Overall, nine dogs were screened for eligibility and included. Screening cardiological examination was performed by the cardiologist of the respective study site. Heart disease was classified as preclinical (occult) or clinical (overt) dilated cardiomyopathy (DCM) according to published guidelines by John D. Bonagura et al. (JVC 2022). Diagnosis and staging of DCM was confirmed by an independent cardiologist. Two dogs were classified as occult DCM (case 100-001 and case 100-004; NYHA class 2/B2; Gerhard Wess JVC 2022) and two dogs as overt DCM (case 100-006 and case 100-007; NYHA Class 3/stage C; Gerhard Wess JVC 2022). The remaining five dogs suffered from various other cardiac disorders and were therefore excluded from the study analysis.

Typical echocardiographic findings in dogs with DCM include increased left ventricular systolic and end-diastolic dimensions, decreased fractional shortening, and increased E-point septal separation. Diastolic left ventricular filling can be assessed noninvasively by pulsed Doppler echocardiography. The transmitral flow (TMF) patter is classified either as restrictive or as nonrestrictive (normal TMF, impaired relaxation, and pseudonormal pattern). In people and dogs, the restrictive TMF pattern correlates well with high left ventricular filling pressure and poor prognosis (Michele Borgarelli et al; JVIM 2006). Borgarelli et al. reported a median survival time in dogs with overt DCM (NYHA 3/stage C) and a restrictive TMF pattern of 80 days (95% CI 42-193 days) when optimally treated with angiotensin-converting enzyme inhibitor (ACE-I), furosemide, and digoxin. In addition, cardiac biomarkers such as N-terminal pro-B-type natriuretic peptide (NT-proBNP) and cardiac troponin I (cTnI) can be used for DCM screening (John D. Bonagura et al.; JVC 2022; Gerhard Wess JVC 2022).

The two dogs classified with occult DCM (case 100-001 and case 100-004) were treated with velagliflozin (oral dose of 0.05 mg/kg bodyweight/day) only till D180 and the echocardiographic values did not alter. Additionally, both dogs did not develop an adverse event during the study period of 180 days and the blood values (hematology and biochemistry) remained within normal. In agreement with previous published data (Michele Borgarelli et al; JVIM 2006) both dogs were still alive at the end of the study.

Case 100-006 is a 10-year-old, intact male, 25.8 kg, mixed breed dog, diagnosed with overt DCM and first onset of congestive heart failure. According to the owner, general condition and exercise tolerance was poor. At the time of the screening the dog did not receive any medication. Case 100-006 was treated until D45 with velagliflozin (oral dose of 0.05 mg/kg bodyweight/day) only. Due to natural progression of the cardiac disease torasemide (diuretic) was added at D45 using a very low dosage (dose 0.08 mg/kg/day—minimal dose recommended by the manufacturer: 0.13 mg/kg bodyweight/day). The cardiac disorder progressed further, and the dog developed atrial fibrillation in addition. After a treatment period of 68 days an adverse event (severe weakness, tachypnea, reduced perfusion, development of atrial fibrillation, and abdominal distension due to ascites) occurred and the dog terminated the study due to progression of the heart disease. The dog was euthanized subsequently at day 69. Clinical signs, cardiac biomarkers (cTnI and NT-proBNP), as well as echocardiographic measurements (severe left atrial and left ventricular enlargement, mild decreased contractility, and restrictive TMF pattern) indicated an advanced stage of the disease at time of inclusion. The patient reached the end of study after 69 days which is in agreement with the reported survival times of DCM dogs receiving standard treatment. This despite the fact, that case 100-006 did not receive any cardiac protective treatment (such as ACE inhibitors) or positive inotropic support (such as digoxin or pimobendan) and only a very low dosage of diuretic. Blood glucose and electrolytes (Na and K) remained within normal limits during the entire study duration. Creatinine revealed a mild increase and urea a moderate increase. These changes might be due to a slight diuretic effect of velagliflozin, decrease in cardiac output due to the DCM, or a combination of these two factors. NT-proBNP normalized during the first 45 days of the study indicating a decrease in left ventricular filling pressure. This finding is supported by a decrease in the normalized left ventricular internal diastolic diameter (LVIDDN). Cardiac TnI increased during the study period as a result of an ongoing progression of the disease. The body weight did not reveal any significant changes.

TABLE 1
Cardiac biomarkers at study visits

| | Screening | | |
|---|---|---|---|
| | visit | | |
| | (D −7 | Visit 2 | End of Study |
| Cardiac biomarkers | to −1) | (D 45) | (D 69) |
| NT-proBNP (pmol/l) | 708.00 | 197.50 | Not measured |
| (n < 500 pmol/l) | | | |
| cTnI (ng/ml) (n < 0.08 ng/ml) | 0.11 | 0.54 | Not measured |

N-terminal pro-B-type natriuretic peptide (NT-proBNP) and cardiac troponin I (cTnI)

TABLE 2
Echocardiographic parameters at study visits

| | Screening visit | Visit 2 |
|---|---|---|
| | (D −7 to −1) | (D 45) |
| Echocardiographic parameters | | |
| LA/Ao (n < 1.5) | 3.08 | 2.69 |
| MV E/A (restrictive > 2.0) | 2.84 | 2.58 |
| Echocardiographic values normalized | | |
| LVIDDN (n < 1.65) | 2.62 | 2.37 |
| EDVI (ml/m2) (n < 100 ml/m2) | 280.19 | 225.31 |
| ESVI (ml/m2) (n < 50 ml/m2) | 147.93 | 135.74 |
| EPSS mm (n < 7 mm) | 13.1 | 19.9 |
| FS % (n > 20%) | 24.36 | 20.25 |

LA (Left atrium dimension measured as right parasternal short-axis); Ao (Aortic root diameter); LA/Ao, left atrium to aorta ratio; LVIDD (Left ventricular internal diameter end diastole); N normalized values according to body surface and body weight; EDVI Left ventricular (LV) end-diastolic volume indexed to body surface area; ESVI Left ventricular (LV) end-systolic volume indexed to body surface area; EPSS (E-point to Septal Separation); FS (fractional shortening)

Case 100-007 is a 7-year-old, intact male, 26 kg, mixed breed dog, diagnosed with overt DCM and first onset of congestive heart failure. According to the owner general condition and exercise tolerance was poor. At the time of the screening the dog did not receive any medication.

Case 100-007 was treated until D45 (43 days) with the velagliflozin (oral dose of 0.05 mg/kg bodyweight/day) only. Due to natural progression of the cardiac disease torasemide (diuretic) was added at D45 (dose: 0.48 mg/kg bodyweight/day). Clinical signs, cardiac biomarker (cTnI), as well as echocardiographic measurements (severe left atrial and left ventricular enlargement, decreased contractility, and restrictive TMF pattern) indicated an advanced stage of the disease. The patient has reached the regular D90 visit without developing an adverse event. This despite the fact, that case 100-007 did not receive any cardiac protective treatment (such as ACE inhibitors) or positive inotropic support (such as digoxin or pimobendan). At D101 the owner reported a worsening of the dog's condition. The unscheduled visit revealed progression of the DCM with development of ascites. Subsequently, spironolactone was added to the current treatment to enhance diuresis. Spironolactone was classified as a prohibited treatment in the study protocol. Therefore, case 100-007 did reach end of study at D101. Blood glucose normalized after initiation of the study drug. Electrolytes (Na and K) and NT-proBNP remained until and including D90 visit within normal limits. At study end D101 creatinine and urea revealed a mild increase. These changes might be due to a slight diuretic effect of velagliflozin, decrease in cardiac output due to the DCM, addition of diuretic treatment (torasemide), or a combination of these three factors. Cardiac TnI decreased significantly at D45 indicating a diminished loss of cardiac myocytes. Thereafter, cTnI increased moderately at D90 as a result of an ongoing progression of the disease. At the end of the study NT-proBNP was elevated due to progression of the disease. Left atrial size as well as left ventricular diastolic and systolic dimensions decreased progressively over the first 90 days of the study. The TMF pattern convened during the first 42 days of the study from a restrictive to a nonrestrictive pattern indicating a decrease in left ventricular diastolic filling pressure. Due to fusion of the early and late diastolic transmitral flow at D90 visit the TMF pattern could not be determined. Left ventricular hypokinesis determined by FS %, EPSS, and ESVI worsened over the first 90 days of the study due to progression of the DCM and lack of pharmacological positive inotropic support. The body weight did not reveal any significant changes.

TABLE 3
Cardiac biomarkers at study visits

| | Screening | | | |
|---|---|---|---|---|
| | visit | | | End of |
| | (D −7 | Visit 2 | Visit 3 | study |
| Cardiac biomarkers | to −1) | (D 45) | (D 90) | (D 101) |
| NT-proBNP (pmol/l) | 13.2 | 103.25 | 424.8 | 735.26 |
| (n < 500 pmol/l) | | | | |
| cTnI (ng/ml) (n < 0.08 | 0.27 | 0.09 | 0.17 | not measured |
| ng/ml) | | | | |

N-terminal pro-B-type natriuretic peptide (NT-proBNP) and cardiac troponin I (cTnI)

TABLE 4
Echocardiographic parameters at study visits

| | Screening | | |
|---|---|---|---|
| | visit | | |
| | (D −7 | Visit 2 | Visit 3 |
| | to −1) | (D 45) | (D 90) |
| Echocardiographic parameters | | | |
| LA/Ao (n < 1.5) | 2.71 | 2.6 | 2.44 |
| MV E/A (restrictive > 2.0) | 2.78 | 1.87 | nd (E/A fusion) |
| Echocardiographic | | | |
| values normalized | | | |
| LVIDDN (n < 1.65) | 2.6 | 2.57 | 2.51 |
| EDVI (ml/m2) (n < 100 ml/m2) | 273.4 | 264.71 | 251.37 |
| ESVI (ml/m2) (n < 50 ml/m2) | 167.59 | 180.79 | 157.26 |
| EPSS mm (n < 7 mm) | 13.7 | 15.9 | 18.9 |
| FS % (n > 20%) | 19.3 | 15.4 | 18.45 |

LA (Left atrium dimension measured as right parasternal short-axis); Ao (Aortic root diameter); LA/Ao, left atrium to aorta ratio; LVIDD (Left ventricular internal diameter end diastole); N normalized values according to body surface and body weight; EDVI Left ventricular (LV) end-diastolic volume indexed to body surface area; ESVI Left ventricular (LV) end-systolic volume indexed to body surface area; EPSS (E-point to Septal Separation); FS (fractional shortening)

### Summary

The two dogs classified with preclinical (occult) DCM were treated with velagliflozin only till D180 and the echocardiographic values did not alter. Additionally, both dogs did not develop an adverse event during the study period of 180 days and their blood values remained within normal.

The two dogs with overt DCM were treated until D45 with velagliflozin only. At D45, both dogs revealed an improvement in left atrial and left ventricular size, and one of the cardiac biomarkers. These findings are unexpected due to withhold of any cardioprotective or positive inotropic treatment. Case 100-007 showed in addition an improvement of the TMF pattern from restrictive to nonrestrictive (MV E/A-ratio) demonstrating a decrease in left ventricular filling pressure (elevated left ventricular filling pressure is an important negative prognostic indicator in humans and dogs). Left ventricular contractility remained poor, since the dogs did not receive any positive inotropic drug. Both dogs showed a mild, however clinically not relevant increase in creatinine and urea. In case 100-007 this increase was transient. The increase in renal parameters might be due to a slight diuretic effect of the study drug, decrease in cardiac output due to the DCM, or a combination of these two factors. Also, both dogs might have some age-related renal changes aggravating the increase in urea und creatinine. Despite their advanced stage of the cardiac disease and receiving velagliflozin and later torasemide only, both dogs with overt DCM showed a regular course of the disease indicating a high effectiveness of velagliflozin with regard to survival time. This even though case 100-006 did receive a very low dosage of torasemide only.

### Conclusion

There was no observed disease progression in case of the two dogs classified with preclinical (occult) DCM and treated with velagliflozin. Despite the fact that they did not receive standard of care treatment both dogs classified with overt DCM and treated with velagliflozin showed survival times comparable to dogs receiving standard of care treatment. This was also accompanied by improvements in cardiac size (LA/AO-ratio, LVIDDN, and LVEDI) and cardiac biomarkers. These findings clearly demonstrate the high effectiveness of velagliflozin with regard to treatment outcome and survival times.

### Example 4 Exploratory Laboratory Study in Dogs with Asymptomatic/Preclinical Myxomatous Mitral Valve Disease (MMVD)

Myxomatous mitral valve disease (MMVD) is the most common acquired cardiac disorder in dogs, accounting for approximately 75% of cases of heart disease in dogs (Buchanan J W., Adv Vet Sci Comp Med. 1977; 21: 75-106). For classification of MMVD the American College of Veterinary Internal Medicine (ACVIM) guidelines are commonly used (Keene B W et al., J Vet Intern Med. 2019; 33: 1127-1140). Dogs with cardiac enlargement that have not yet developed signs of congestive heart failure are considered to be in asymptomatic/preclinical ACVIM stage B2.

Echocardiographic findings in dogs with MMVD stage B2 include degenerative valvular changes, increased left atrial dimension (LA/AO-ratio≥1.6), and increased left ventricular end-diastolic dimensions normalized to body weight (LVIDDN≥1.7) (Keene et al., 2019). For evaluation of survival time a severity score (MINE score) based on the echocardiographic variables above, the cardiac contractility (fractional shortening, FS %), and transmitral peak E-wave velocity was initiated (Vezzosi T et al., J Vet Intern Med. 2021, 35(3):1238-1244). The MINE score is associate with the survival time and provides therefore prognostic information.

Stage B2 dogs are of particular interest since they are likely to benefit substantially from treatment. So far, only pimobendan demonstrated a prolongation of the preclinical period (Boswood A et al., J Vet Intern Med. 2016; 30: 1765-1779).

The objective of this study was to evaluate velagliflozin, a sodium-glucose co-transporter-2 inhibitor, as a treatment for dogs with asymptomatic/preclinical ACVIM stage B2 MMVD. Three Beagle dogs previously diagnosed with this condition by a veterinary cardiologist were included in the study.

Baseline assessments included body weight measurements, veterinary examinations, echocardiographs, urinalysis, blood collections for hematology, biochemistry, BHB, NT-proBNP, cTnI, and erythropoietin analysis as well as radiographs to assess heart size, pulmonary edema, and congestion. On study Day 0, once daily oral administration of velagliflozin (0.05 mg/kg bodyweight) was initiated. Veterinary examinations, urinalysis, and blood collections for analysis of the above-mentioned parameters were repeated on Days 32, 59, and 91. Echocardiographs were repeated on Days 23, 58, and 93. General health observations are conducted twice daily, and body weights measured approximately every 3 weeks.

### Echocardiograph Data:

Over a velagliflozin treatment period of 94 days, improvements (i.e., decreases) have been observed in two echocardiography parameters which are commonly used to assess MMVD severity and progression: left atrial-to-aortic ratio (LA/Ao) and left ventricular internal diameter in diastole normalized for body weight (LVIDDN).

The first dog (Azul) showed a 17.3% decrease from baseline in LA/Ao ratio on Day 58. Although an increase was observed on Day 93, her value remained 1.08% below baseline. The second subject (Birdie) showed a reduction of 12.73% from baseline on Day 93. The third subject (Shelby) had not shown a reduction in LA/Ao ratio from baseline.

LVIDDN was reduced by 3.56% at Day 58 for Azul; however, returned to just above baseline levels (+0.81%) by Day 93. Both Birdie and Shelby showed LVIDDN values which were reduced from baseline at Day 23, 58, and 93, with values on Day 93 reduced by 5.25% and 3.54%, respectively. It should be noted that changes in body weight affects LVIDDN. As all subjects experienced a decrease in body weight prior to the Day 23 echo measurement, values from this time point should be interpreted with caution.

Additionally, the “The Mitral Insufficiency Echocardiographic” score, a severity classification of myxomatous mitral valve disease (MMVD) in dogs (MINE score) was calculated (Vezzosi et al., 2021). This score as an easy-to-use echocardiographic classification of severity of MMVD based on routinely acquired echocardiographic variables. The hypothesis was that the MINE score is clinically effective since it is associated with median survival time of the animals (see tables 5 and 6 below).

TABLE 5
Severity classification based on the total score
obtained from summation of the single scores

| Severity classification | Total Score |
|---|---|
| Mild | 4-5 |
| Moderate | 6-7 |
| Severe | 8-12 |
| Late Stage | 13-14 |

TABLE 6
Total MINE score evaluation for the three tested dogs

| Animal | Study day | Total MINE score evolution |
|---|---|---|
| Azul | Base line | 7 (Moderate) |
| | Day 93 | 8 (Increase due to increase in FS %, also showed an |
| | | increase in heart rate and cystitis; this elevation |
| | | might be due to the cystitis) |
| Birdie | Base line | 5 (Mild) |
| | Day 93 | 5 (Mild) |
| Shelby | Base line | 7 (Moderate) |
| | Day 93 | 6 (Moderate) (decrease due to improvement in left |
| | | ventricular diameter) |

Bloodwork: Hematology, Biochemistry, BHB, NT-proBNP, cTnI, and EPO:

No clinically significant changes have been noted on hematology, biochemistry, or erythropoietin parameters thus far. Changes in cTnI also do not appear to be showing a clinically significant trend, which is consistent with previous studies that have shown cTnI to have lower discriminatory ability between stages of MMVD and lower predictive value for determining risk of congestive heart failure.

Beginning at baseline, NT-proBNP was notably elevated above the reference range in one subject (Shelby). During the treatment phase, Shelby's NT-proBNP values have consistently decreased at each measurement time point. Although still above the reference range, by Day 91, her value was reduced by 45.6% from baseline. Azul's NT-proBNP values have remained within the reference range but have also showed a downward trend from baseline to Day 59 (76.3% reduction) with a slight increase on Day 91 (45.8% reduction from baseline). BHB was reduced by 10× in 2 subjects (Azul and Birdie) on Day 59; however, returned to baseline levels on Day 91.

### Adverse Events:

Following initiation of treatment, the most frequent abnormal observation has been vomit found within the housing pen. As the animals are group housed, occurrence frequency for individual subjects cannot be determined. The relationship of vomiting to the test product is unknown; however, there was also one instance of vomit in the housing pen noted at baseline. No concurrent treatment interventions have been required for management of vomiting.

Lip smacking/licking, head shaking, grimacing, and salivating have been observed following dosing. Such observations can be attributed to oral administration of a liquid product not contained within a capsule and did not interfere with retention of intended dose volumes

One animal developed a cystitis during treatment (Azul). The animal was treated at discretion of the local veterinarian and recovered without any further issues. Cystitis have been reported as a common adverse event of SGLT2 treatment in humans. Additionally, Azul showed an increase in contractility FS % at D93 and an elevated heart rate. Both of which could be explained by discomfort/abdominal pain caused by the cystitis. It is important to notice that blood glucose and kidney values were not affected during this study, as well as the urinary specific gravity, demonstrating the safety profile of the study medication velagliflozin.

### Summary

All three dogs with asymptomatic/preclinical MMVD ACVIM stage B2 were treated orally until the interim analysis at D94 with 0.05 mg/kg bodyweight velagliflozin once daily only. None of the study dogs developed congestive heart failure (CHF) until D94. Aside from mild gastrointestinal signs (vomiting, salivation, smacking, etc.) one animal developed a bacterial cystitis due to glucosuria (Azul). Glucosuria was noted in all three dogs demonstrating pharmacodynamic effectiveness of the study drug velagliflozin. However, no changes in blood glucose, renal function, or serum electrolytes were noted at any time in any animal.

So far, all three dogs showed a regular course of the disease and were still alive at the time of the interim analysis.

With regard to left atrial size Azul showed no change in the LA/AO-ratio, Birdie revealed a normalization of the left atrial size, and Shelby demonstrated a slight increase (worsening) of the LA/AO-ratio. The reported day-to-day intraobserver repeatability by Visser et al. (J Vet Intern Med. 2019; 33: 1909-1920) for LA/AO-ratio revealed a within subject coefficient of variation of 11.0% and a 95% repeatability coefficient of 0.44. Changes for Azul and Shelby were therefore due to day-to-day variability. The change in left atrial size of Birdie showed an improvement beyond this variability indicating a clinically relevant amelioration.

With regard to normalized left ventricular inner diameter (LVIDDN), Azul showed no change in LVIDDN, whereas Birdie and Shelby demonstrated a slight decrease (improvement). The reported day-to-day intraobserver repeatability by Visser et al. (J Vet Intern Med. 2019; 33: 1909-1920) for LVIDDN revealed a within subject coefficient of variation of 5.9% and a 95% repeatability coefficient of 0.23. Changes for all three dogs were therefore due to day-to-day variability.

As reflected by the MINE score Birdie revealed a mild stage of the disease at baseline and at D93, Shelby a moderate stage at baseline and D93, and Azul a moderate stage at baseline and a severe stage at D93. The worsening of the MINE score in Azul was due to an increase in fractional shortening FS %. According to the law of Frank Starling the FS % can be influenced by the preload or by a change in the sympathetic tone. The preload can be assessed by the left atrial size. Due to the unaltered LA/AO ratio a change in the preload as a cause for the increased FS can be excluded. At that time Azul revealed a bacterial cystitis. Cystitis in general is usually accompanied by abdominal discomfort and pain and therefore an elevated sympathetic tone. At D93 Azul revealed a slightly higher heart rate than at baseline supporting the presence of an increased sympathetic tone as a possible cause for the worsened MINE score.

### CONCLUSION

During the oral treatment of all three dogs with asymptomatic/preclinical MMVD ACVIM stage B2 with 0.05 mg/kg bodyweight velagliflozin once daily only until the interim analysis at D94 none of the study dogs developed congestive heart failure (CHF). Overall, the measured data demonstrate stable disease states with signs of improvement in some of the key diagnostic parameters used to assess MMVD.

### REFERENCES

(1) Bonagura J D et al., J Vet Cardiol 2022, 40: 15-50
(2) Borgarelli M et al; J Vet Intern Med 2006, 20(1): 104-110
(3) Boswood A et al., J Vet Intern Med. 2016; 30: 1765-1779
(4) Buchanan J W, Adv Vet Sci Comp Med. 1977; 21: 75-106
(5) Keene B W et al., J Vet Intern Med. 2019; 33: 1127-1140
(6) Lin Y et al., J Am Heart Assoc 2021, 10: e019274
(7) Matsumura K et al., Cardiovascular Ultrasound 2019, 17(1): 26
(8) Nishinarity R et al., J Am Heart Assoc 2021, 10:e017483
(9) Santos-Gallego C G et al., J American College Cardiol 2019, 73(15): 1931-1944
(10) Silva Custodio Jr J et al., Heart Failure Reviews 2018, 23(3): 409-418
(11) US 2011/098240
(12) US 2015/164856
(13) US 2016/000816
(14) US 2017/266152
(15) US 2019/076395
(16) U.S. Pat. No. 10,537,570
(17) Vezzosi T et al., J Vet Intern Med. 2021, 35(3): 1238-1244
(18) Visser et al., J Vet Intern Med. 2019; 33: 1909-1920
(19) Wess G, J Vet Cardiol 2022, 40: 51-68
(20) WO 2021/092341

1. A method of prevention and/or treatment of one or more cardiac diseases in a non-human mammal excluding a feline, the method comprising administering to the non-human mammal a pharmaceutical composition comprising one or more SGLT-2 inhibitors or a pharmaceutically acceptable form thereof to the non-human mammal.

2. The method of claim 1, wherein the one or more cardiac diseases ae is selected from the group consisting of heart failure; congestive heart failure; asymptomatic/preclinical/occult heart failure; heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); and aortic stenosis (valvular, supravalvular and/or subvalvular).

3. The method of claim 1, wherein the one or more cardiac diseases is selected from the group consisting of (myxomatous) mitral valve disease [(M)MVD]; clinically overt (myxomatous) mitral valve disease [(M)MVD]; asymptomatic/preclinical/occult (myxomatous) mitral valve disease [(M)MVD]; dilated cardiomyopathy (DCM); clinically overt dilated cardiomyopathy (DCM); and asymptomatic/preclinical/occult dilated cardiomyopathy (DCM).

4. The method of claim 1, wherein the one or more cardiac diseases is selected from the group consisting of mitral valve disease [(M)MVD]; clinically overt (myxomatous) mitral valve disease [(M)MVD]; and asymptomatic/preclinical/occult (myxomatous) mitral valve disease [(M)MVD].

5. The method of claim 1, wherein the one or more SGLT-2 inhibitors comprises a glucopyranosyl-substituted benzene derivative.

6. The one or more SGLT-2 inhibitors or pharmaceutically acceptable forms thereof for the use according to any one of claims 1 to 5 The method of claim 1, wherein the one or more SGLT-2 inhibitors is selected from the group consisting of:
(1) a glucopyranosyl-substituted benzene derivative of the formula (1) 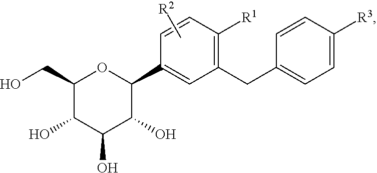
wherein R¹ denotes cyano, Cl or methyl;
R² denotes H, methyl, methoxy or hydroxy and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2): 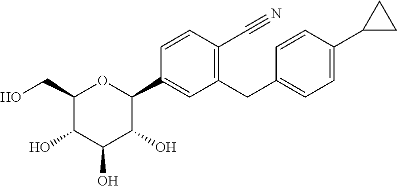 [SMILES: N#Cc1ccc([C@@H]2O[C@H](CO)[C@@H](O)[C@H](O)[C@H]2O)cc1Cc1ccc(C2CC2)cc1]
(3) Dapagliflozin, represented by formula (3): 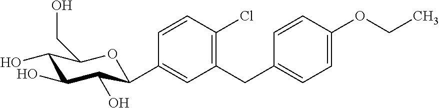
(4) Canagliflozin, represented by formula (4): 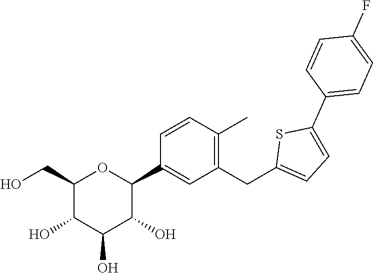 [SMILES: Cc1ccc([C@@H]2O[C@H](CO)[C@@H](O)[C@H](O)[C@H]2O)cc1Cc1ccc(-c2ccc(F)cc2)s1]
(5) Empagliflozin, represented by formula (5): 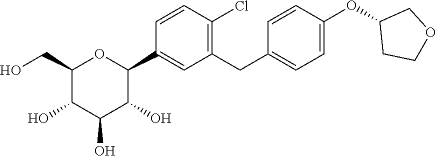 [SMILES: OC[C@H]1O[C@@H](c2ccc(Cl)c(Cc3ccc(O[C@H]4CCOC4)cc3)c2)[C@H](O)[C@@H](O)[C@@H]1O]
(6) Luseogliflozin, represented by formula (6): 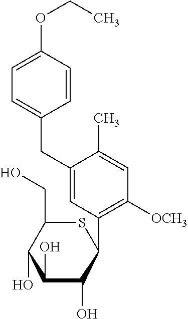
(7) Tofogliflozin, represented by formula (7): 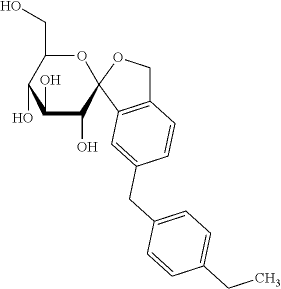
(8) Ipragliflozin, represented by formula (8): 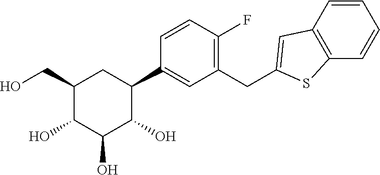 [SMILES: OC[C@H]1C[C@@H](c2ccc(F)c(Cc3cc4ccccc4s3)c2)[C@H](O)[C@@H](O)[C@@H]1O]
(9) Ertugliflozin, represented by formula (9): 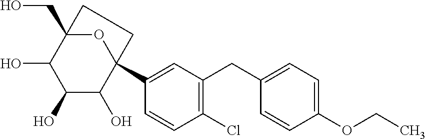 [SMILES: CCOc1ccc(Cc2cc([C@]34CC[C@](CO)(O3)C(O)[C@H](O)C4O)ccc2Cl)cc1]
(10) Atigliflozin represented by formula (10): 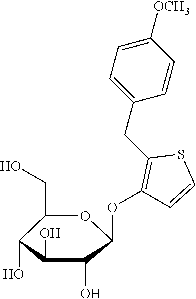
(11) Remogliflozin, represented by formula (11): 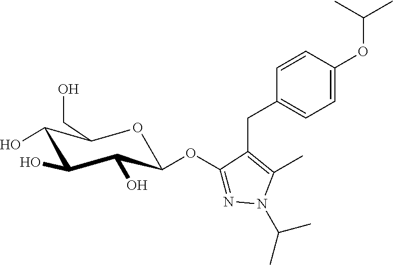
(11A) Remogliflozin etabonate, represented by formula (11A): 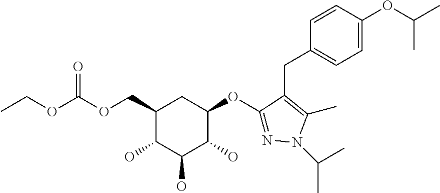 [SMILES: CCOC(=O)OC[C@H]1C[C@@H](Oc2nn(C(C)C)c(C)c2Cc2ccc(OC(C)C)cc2)[C@H](O)[C@@H](O)[C@@H]1O]
(12) a thiophene derivative of the formula (12) 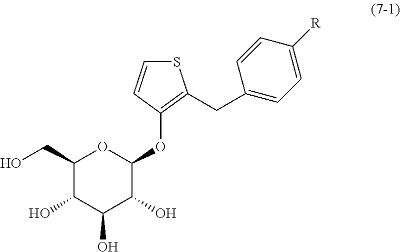
wherein R denotes methoxy or trifluoromethoxy;
(13) 1-(β-D-glucopyranosyl)-4-methyl-3-[5-(4-fluorophenyl)-2-thienylmethyl]benzene, represented by formula (13); 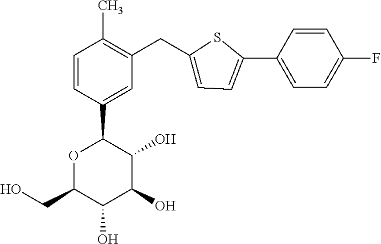 [SMILES: Cc1ccc([C@@H]2O[C@H](CO)[C@@H](O)[C@H](O)[C@H]2O)cc1Cc1ccc(-c2ccc(F)cc2)s1]
(14) a spiroketal derivative of the formula (14): 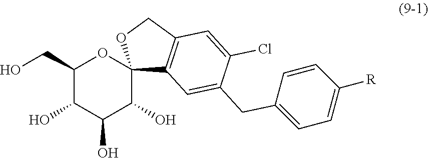
wherein R denotes methoxy, trifluoromethoxy, ethoxy, ethyl, isopropyl or tert. butyl;
(15) a pyrazole-O-glucoside derivative of the formula (15) 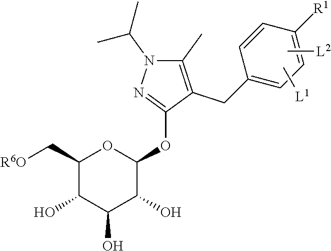
wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin represented by formula (16): 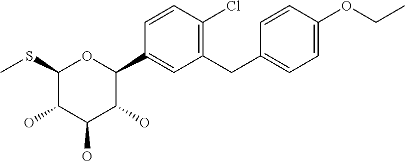 [SMILES: CCOc1ccc(Cc2cc([C@@H]3O[C@H](SC)[C@@H](O)[C@H](O)[C@H]3O)ccc2Cl)cc1]
(17) Sergliflozin, represented by formula (17): 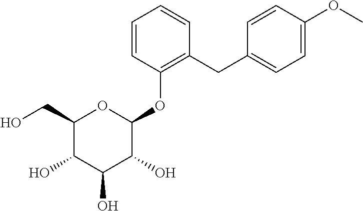 [SMILES: COc1ccc(Cc2ccccc2O[C@@H]2O[C@H](CO)[C@@H](O)[C@H](O)[C@H]2O)cc1]
(18) a compound represented by formula (18): 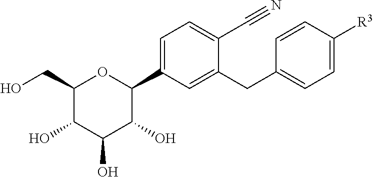
wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano,
or a derivative thereof wherein one or more hydroxyl groups of the β-D-glucopyranosyl group are acylated with groups selected from (C₁₋₁₈alkyl)carbonyl, (C₁₋₁₈alkyl)oxycarbonyl, phenylcarbonyl and phenyl-(C₁₋₃-alkyl)-carbonyl;
(19) Bexagliflozin, represented by formula (19): 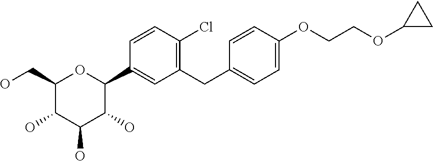 [SMILES: OC[C@H]1O[C@@H](c2ccc(Cl)c(Cc3ccc(OCCOC4CC4)cc3)c2)[C@H](O)[C@@H](O)[C@@H]1O]
(20) Janagliflozin, represented by formula (20): 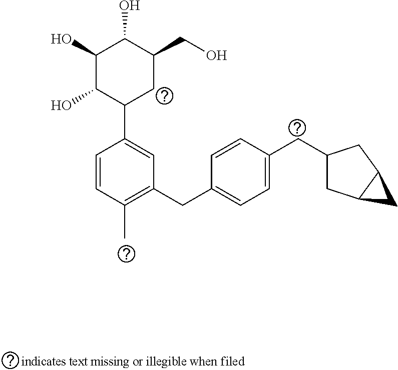 [SMILES: Cc1ccc(C2C[C@H](CO)[C@@H](O)[C@H](O)[C@H]2O)cc1Cc1ccc(CC2C[C@@H]3C[C@@H]3C2)cc1]
(21) Rongliflozin, represented by formula (21): 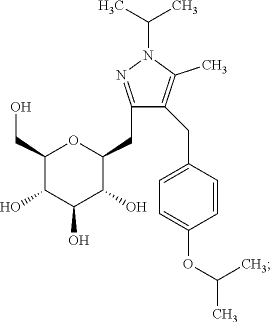 [SMILES: Cc1c(Cc2ccc(OC(C)C)cc2)c(C[C@@H]2O[C@H](CO)[C@@H](O)[C@H](O)[C@H]2O)nn1C(C)C]
and
(22) Wanpagliflozin.

7. The method of claim 1, wherein the pharmaceutically acceptable form thereof is a crystalline complex between the one or more SGLT2 inhibitors and one or more amino acids, such as proline.

8. The method of claim 1, wherein the non-human mammal is a dog in need of such prevention and/or treatment.

9. The method of claim 1, wherein the one or more SGLT-2 inhibitors is administered orally, parenterally, intravenously, subcutaneously or intramusculary.

10. The method of claim 1, wherein the one or more SGLT-2 inhibitors is administered at a dose of 0.01 mg/kg bodyweight to 10 mg/kg bodyweight per day, or at a dose of 0.01 mg/kg bodyweight to 5 mg/kg bodyweight per day, or at a dose of 0.01 mg/kg bodyweight to 4 mg/kg bodyweight per day, or at a dose of 0.01 mg/kg bodyweight to 3 mg/kg bodyweight per day, or at a dose of 0.01 mg/kg bodyweight to 2 mg/kg bodyweight per day, or at a dose of 0.01 mg/kg bodyweight to 1 mg/kg bodyweight per day, or at a dose of 0.01 mg/kg bodyweight to 0.5 mg/kg bodyweight per day, or at a dose of 0.01 mg/kg bodyweight to 0.3 mg/kg bodyweight per day.

11. The method of claim 1, wherein the one or more SGLT2 inhibitors or pharmaceutically acceptable forms thereof is administered once per day or twice per day.

12. The method of claim 1, wherein the one or more SGLT-2 inhibitors consists of velagliflozin administered at once or twice per day at a dose of 0.01 mg/kg bodyweight to 1 mg/kg bodyweight, or at a dose of 0.01 mg/kg bodyweight to 0.5 mg/kg bodyweight, or at a dose of 0.01 mg/kg bodyweight to 0.3 mg/kg bodyweight, or once daily at a dose of 0.05 mg/kg bodyweight.

13. The one or more SGLT2 inhibitors or pharmaceutically acceptable forms thereof for the use according to claim 12 The method of claim 1, wherein the one or more SGLT-2 inhibitors consists of velagliflozin orally administered once daily at a dose of 0.01 mg/kg bodyweight to 0.3 mg/kg bodyweight, or at a dose of 0.05 mg/kg bodyweight.

14. The method of claim 1, wherein the one or more SGLT-2 inhibitors is administered before, after or concomitantly with administering one or more other active pharmaceutical ingredients selected from the group consisting of one or more diuretics, such as furosemide, torasemide or spironolactone; one or more beta-blockers, such as atenolol or propranolol; one or more calcium-channel blockers, such as amlodipine and diltiazem; one or more ACE inhibitors, such as benazepril, ramipril or enalapril; one or more angiotensin receptors blockers, such as telmisartan; one or more antiarrhythmic agents, such as flecainide; one or more platelet agglutination inhibitors, such as clopidogrel; one or more nonsteroidal anti-inflammatory drugs (NSAIDs), such as aspirin; one or more anticoagulants, such as Coumarins (vitamin K antagonists), (low molecular weight) heparin, synthetic pentasaccharide inhibitors of factor Xa, as well as direct factor Xa inhibitors and/or direct thrombin inhibitors; and/or one or more calcium-channel sensitizers and/or positive inotropes, such as pimobendan and/or digitalis alkaloids.

15. The method of claim 1, wherein the administration provides a preventive and/or therapeutic effect that includes one or more of the following clinical and/or biochemical parameters:
improved cardiometabolic efficiency, characterized by an increased ratio of [cardiac output/metabolic substrate consumed] and/or characterized by an increased ratio of [cardiac output/oxygen consumed];
increase of the production of ketone bodies in the liver, characterized by increased plasma levels of 3-hydroxybutyric acid and/or the corresponding acylcarnitines i.e. hydroxybutyrylcarnitine and increased plasma levels of one or more of the branched-chain amino acids (valine, leucine and isoleucine);
improved cardiac function by achieved reduced pre- and/or afterload, improved arterial wall structure function;
improved echocardiographic parameters, such as decreased LA (Left atrium dimension measured as right parasternal short-axis), LA/Ao (left atrium to aorta ratio; Ao=Aortic root diameter), IVSd (interventricular septal end diastolic dimension, i.e. the thickness of the interventricular septum), and/or LAD (Left atrium measured as right parasternal long-axis), and improved cardiac biomarkers, such as decreased NT-proBNP (N-terminal prohormone of brain natriuretic peptide) and/or decreased cTnI (cardiac Troponin I) and/or increased erythropoietin concentration; as well as improved heart murmur;
delayed onset of different phenotypes of cardiac diseases, such as (M)MVD and/or DCM, at least by 1, 2, 3, 4, 5, 6, 7, 8, 9, 10, 11, 12, 13, 14, 15 or more months, or even stopped progression of different phenotypes of cardiac diseases, such as (M)MVD and/or DCM;
longer time of survival, at least by 1, 2, 3, 4, 5, 6, 7, 8, 9, 10, 11, 12, 13, 14, 15 or more months, and/or delay of next episode of heart failure, at least by 1, 2, 3, 4, 5, 6, 7, 8, 9, 10, 11, 12, 13, 14, 15 or more months, and/or lower level of cardiac mortality and/or morbidity;
improved clinical signs, such as reduced e.g. breathlessness or dyspnea, cough, depression, exercise intolerance, inappetence, syncope, abdominal distention and/or polydipsia;
prolongation of time to heart failure, cardiac death, onset of clinical signs, need for additional concomitant medication, and/or increase in dose of concomitant therapy—diuretics); and
higher quality of life.

16. The method of claim 1, wherein the pharmaceutically acceptable form thereof is a crystalline complex between the one or more SGLT2 inhibitors and one or more amino acids, L-proline and crystalline water.

17. The method of claim 2, wherein the one or more cardiac diseases are characterized by symptoms of heart failure selected from the group consisting of: exercise intolerance, dyspnoea, fatigue, fluid retention and reduced longevity.
